(19) United States
(12) Patent Application Publication
(10) Pub. No.: US 2020/0223809 A1
(43) Pub. Date: Jul. 16, 2020

(54) ANTHRACENE MATERIALS, ORGANIC LIGHT EMITTING DIODES, AND METHOD FOR MANUFACTURING ANTHRACENE MATERIALS

(71) Applicants: Yuan Ze University, Chung-Li (TW); Nichem Fine Technology Co, Ltd., Jhubei City (TW); Wisechip Semiconductor Inc., Zhunan Township (TW); Tetrahedron Technology Corporation, Zhunan Township (TW); Shine Materials Technology Co., Ltd., Kaohsiung City (TW)

(72) Inventors: Tien-Lung Chiu, Chung-Li (TW); Jiun-Haw Lee, Chung-Li (TW); Man-kit Leung, Chung-Li (TW); Chi-Feng Lin, Chung-Li (TW); Zheng-Chen Hsiao, Chung-Li (TW); Yi-Mei Huang, Chung-Li (TW); Bo-An Fan, Chung-Li (TW)

(73) Assignees: Yuan Ze University, Chung-Li (TW); Nichem Fine Technology Co, Ltd., Jhubei City (TW); Wisechip Semiconductor Inc., Zhunan Township (TW); Tetrahedron Technology Corporation, Zhunan Township (TW); Shine Materials Technology Co., Ltd., Kaohsiung City (TW)

(21) Appl. No.: 16/739,953

(22) Filed: Jan. 10, 2020

(30) Foreign Application Priority Data
Jan. 11, 2019 (TW) 108101238

(51) Int. Cl. C07D 277/66; C07D 235/08; C07D 263/57; C07D 271/12; H01L 51/00

(52) U.S. Cl. CPC C07D 277/66; C07D 235/08; C07D 263/57; H01L 51/5056; H01L 51/0072; H01L 51/0052; C07D 271/12

(57) ABSTRACT

An anthracene material, an organic light emitting diode using the same, and a method for manufacturing the same, are provided. The organic light emitting diode includes a substrate, a first conducting layer, a hole transport layer, a light emitting layer, an electron transport layer, and a second conducting layer. The first conducting layer is disposed on the substrate. The hole transport layer is disposed on the first conducting layer. The light emitting layer having the anthracene material is disposed on the hole transport layer. The electron transport layer is disposed on the light emitting layer. The second conducting layer is disposed on the electron transport layer.

### BACKGROUND

### Technical Field

The present invention relates to an anthracene material, an organic light emitting diode using the same, and a method for manufacturing the same. More specifically, the present invention relates to a dianthracenebenzimidazole material, an organic light-emitting diode using the same, and a method for manufacturing the same.

### Related Art

Liquid crystal displays (LCDs) have become mainstream in recent years. For example, LCDs have wide applications in televisions, personal computers, laptops, monitors, mobile phones, digital cameras, and so on. In these applications, the backlight module of an LCD should be a light source with enough brightness and even light distribution so that the LCD can display images normally.

Having advantages such as a wide viewing angle, fast response time, high brightness, low power, and a broad operating temperature range, organic light emitting diodes have gradually become a common luminescent element of backlight modules. Current organic light emitting diodes mainly uses a host-guest system, and theoretically, can reach an internal quantum efficiency of 100% by a suitable phosphorescent guest emitter, so that phosphorescent materials recently have become a developing trend of organic electroluminescent materials.

In the development of blue host materials, host materials must have triplet energy levels greater than or equal to those of guest materials to avoid problems caused by energy loss due to energy return and including low luminous efficiency (also known as current efficiency) and a short lifetime, and therefore, it is a must to have higher triplet energy levels. Furthermore, an emissive layer should be made from material with a good energy level alignment and a high glass transition temperature (Tg) allowing a good thermal stability.

### SUMMARY

The main object of the invention is to provide anthracene materials that feature a blue radiation range, a high glass transition temperature, and a good luminous efficiency.

Another object of the invention is to provide organic light emitting diodes with higher efficiency and a longer lifetime.

Another object of the invention is to provide a manufacturing method of anthracene materials.

The anthracene materials of the invention have the structure of the following formula (1):

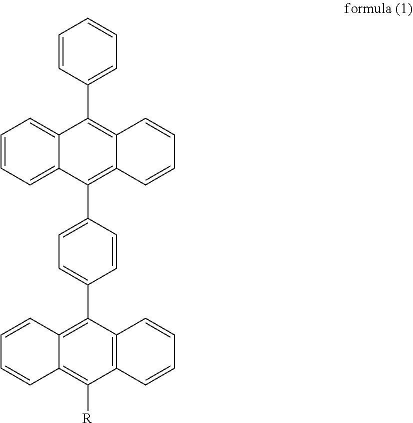

wherein, R is selected from the group consisting of the following groups:

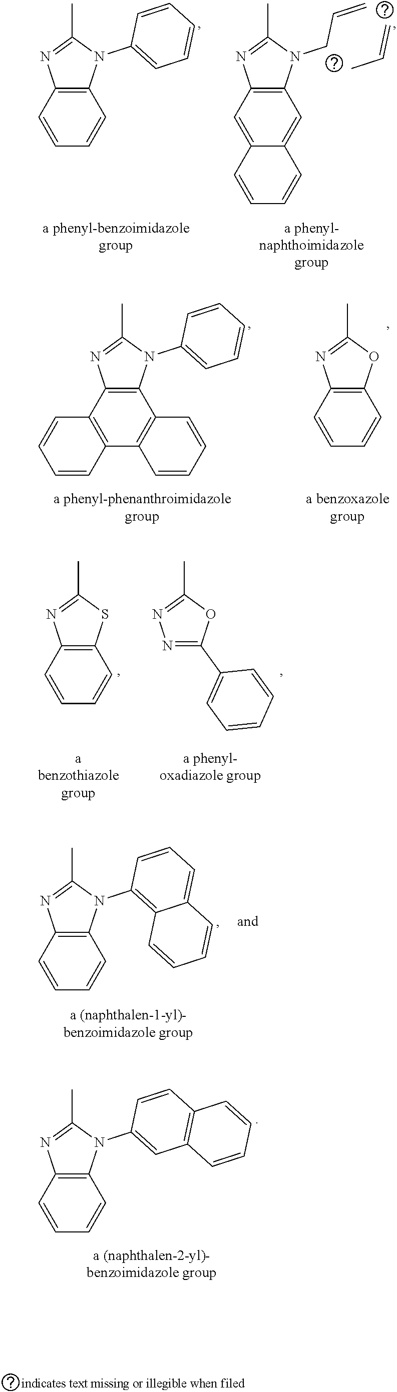

The organic light emitting diodes include a substrate, a first conducting layer, a hole transport layer, a light emitting layer, an electron transport layer, and a second conducting layer. The first conducting layer is disposed on the substrate. The hole transport layer is disposed on the first conducting layer. The light emitting layer is disposed on the hole transport layer, and has the anthracene materials with the structure of the following formula (1):

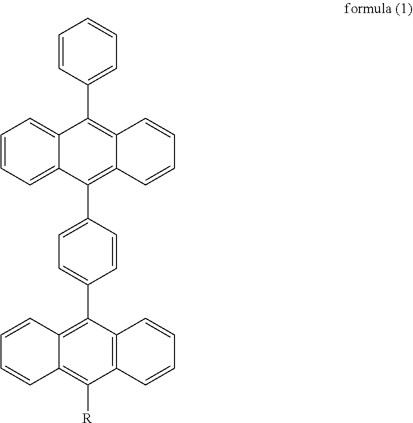

The electron transport layer is disposed on the light emitting layer. The second conducting layer is disposed on the electron transport layer.

wherein, R is selected from the group consisting of the following groups:

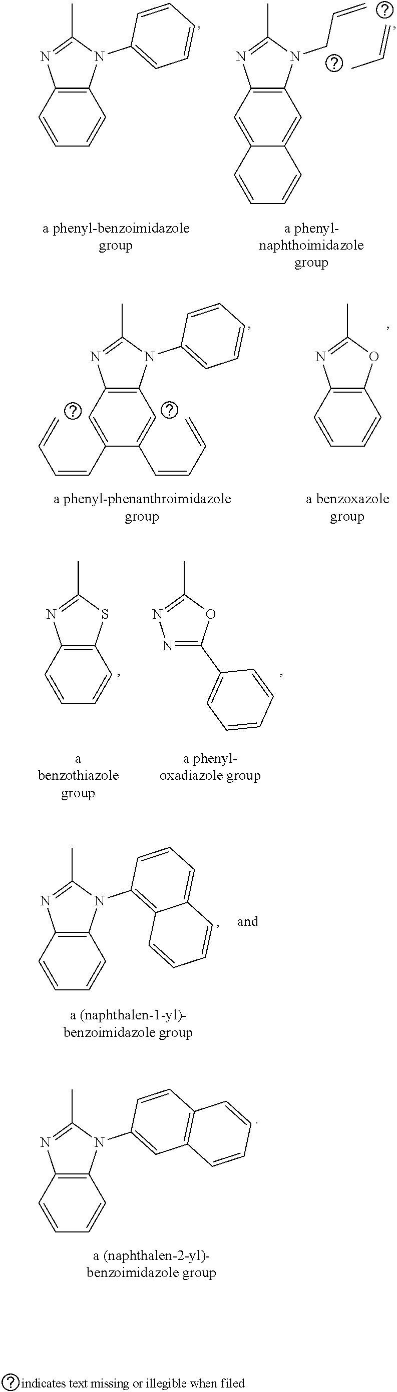

In an embodiment of the invention, the light emitting layer has a thickness of 200 Å.

In an embodiment of the invention, the light emitting layer contains 9,9′-(2-(1-phenyl-1H-benzo[d]imidazol-2-yl)-1,3-phenylene)bis(9H-carbazole) (o-DiCbzBz) as a host and 1-phenyl-2-(10-(4-(10-phenylanthracen-9-yl)phenyl)anthracen-9-yl)-1H-benzo[d]imidazole (dianthracenebenzimidazole (diAnBiz)) as a guest, wherein the o-DiCbzBz is doped with 13 v/v % of the dianthracenebenzimidazole.

In an embodiment of the invention, the light emitting layer is an anode.

In an embodiment of the invention, the hole transport layer includes a hole injection layer and a hole transfer layer disposed on the hole injection layer.

In an embodiment of the invention, the electron transport layer includes an electron transfer layer and an electron injection layer disposed on the electron transfer layer.

In an embodiment of the invention, the method for manufacturing the anthracene materials includes production of 9-(4-bromophenyl)-10-phenylanthracene by the following equation,

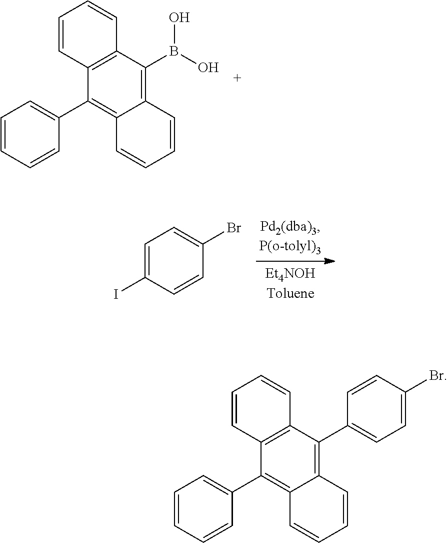 [SMILES: OB(O)c1c2ccccc2c(-c2ccccc2)c2ccccc12 ; Brc1ccc(I)cc1 ; Brc1ccc(-c2c3ccccc3c(-c3ccccc3)c3ccccc23)cc1]

In an embodiment of the invention, the method for manufacturing the anthracene materials includes production of 10-(4-(10-phenylanthracen-9-yl)phenyl)anthracene-9-carbaldehyde by the following equation,

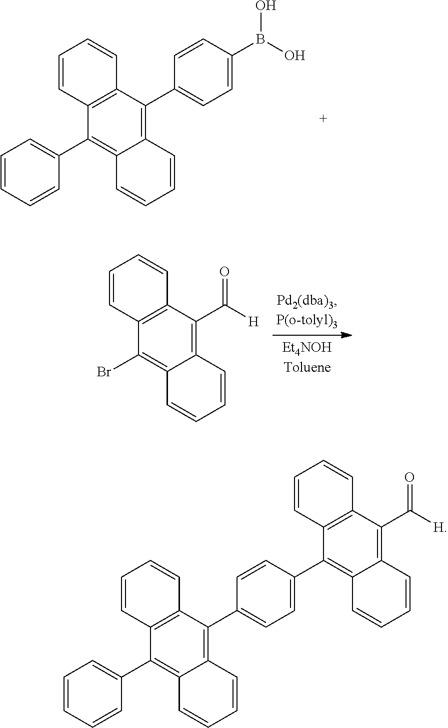

In an embodiment of the invention, the method for manufacturing the anthracene materials includes production of 1-phenyl-2-(10-(4-(10-phenyl)anthracen-9-yl)phenyl)anthracen-9-yl)-1H-benzo[d]imidazole (dianthracenebenzimidazole) by the following equation,

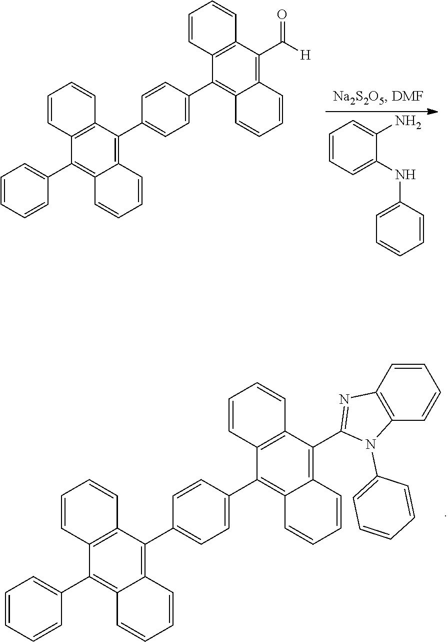 [SMILES: O=Cc1c2ccccc2c(-c2ccc(-c3c4ccccc4c(-c4ccccc4)c4ccccc34)cc2)c2ccccc12 ; Nc1ccccc1Nc1ccccc1 ; c1ccc(-c2c3ccccc3c(-c3ccc(-c4c5ccccc5c(-c5nc6ccccc6n5-c5ccccc5)c5ccccc45)cc3)c3ccccc23)cc1]

### BRIEF DESCRIPTION OF THE DRAWINGS

The patent or application file contains at least one color drawing. Copies of this patent or patent application publication with color drawing will be provided by the USPTO upon request and payment of the necessary fee.

FIGS. 1A and 1B respectively show the results from measurement on thermal properties of diAnBiz and monoBiz.

FIGS. 2A, 2B and FIGS. 2C, 2D respectively show the results from measurement on electrochemical properties of diAnBiz and monoBiz.

FIGS. 3A and 3B respectively show the results from measurement on photophysical properties of diAnBiz and monoBiz.

FIGS. 4A-4E are schematic representations of a TTA-UC mechanism, and testing on PdOEP and diAnBiz.

FIG. 5 is a schematic representation of an embodiment of the organic light emitting diodes of the invention.

FIG. 6 is a schematic representation of a different embodiment of the organic light emitting diodes of the invention.

FIGS. 7A and 7B show the results from testing on energy level for films.

FIGS. 8A-8G show the results from testing of the organic light emitting diodes with the different compounds as the light emitting layer.

FIGS. 9A-9G show the results from testing of the organic light emitting diodes with diAnBiz of various thicknesses as the light emitting layer.

FIG. 10 is a diagram for element configuration and energy level.

FIGS. 11A-11G show the results from testing of the organic light emitting diodes with diAnBiz of various doping ratios as light emitting layers.

### DETAILED DESCRIPTION

The inventive technique synthesizes a series of anthracene materials by using di-anthracene (diAn for short below) as a group of hole transport properties and benzimidazole (Biz for short below), for example, as a group of electron transport properties. Because the diAn group has a high triplet energy level and the Biz group, for example, has a good thermal stability, the anthracene materials have the potential to serve as the host materials of phosphorescent organic light-emitting diodes (PHOLEDs). Furthermore, because of its structure, the diAn group further helps keep an intermolecular distance.

More specifically, the anthracene materials of the invention have the structure of the following formula (1):

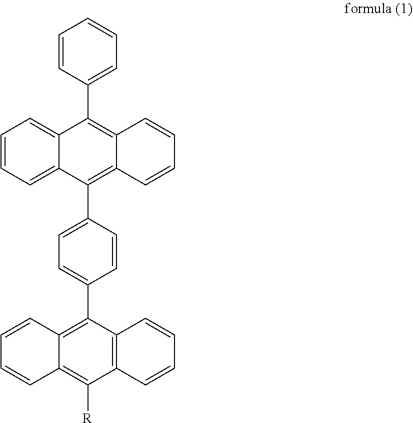

wherein, R is selected from the group consisting of the following groups:

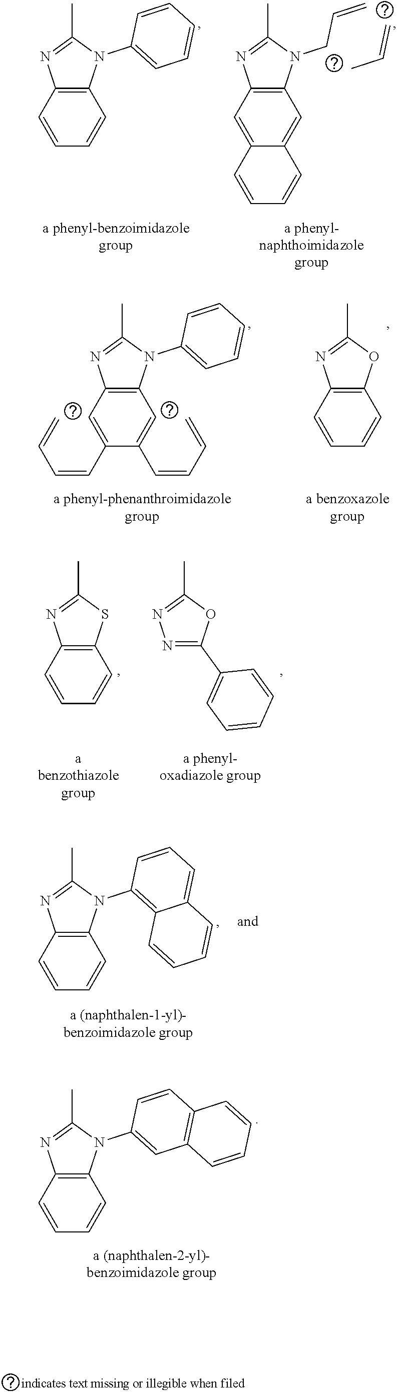

More specifically, the anthracene materials of the invention include, for example, the following.

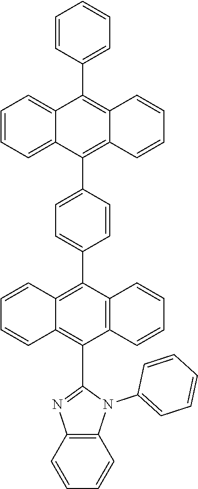 [SMILES: c1ccc(-c2c3ccccc3c(-c3ccc(-c4c5ccccc5c(-c5nc6ccccc6n5-c5ccccc5)c5ccccc45)cc3)c3ccccc23)cc1]

1-phenyl-2-(10-(4-(10-phenylanthracen-9-yl)phenyl)anthracen-9-yl)-1H-benzo[d]imid azole (dianthracenebenzimidazole).

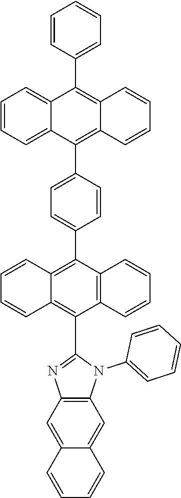 [SMILES: c1ccc(-c2c3ccccc3c(-c3ccc(-c4c5ccccc5c(-c5nc6cc7ccccc7cc6n5-c5ccccc5)c5ccccc45)cc3)c3ccccc23)cc1]

1-phenyl-2-(10-(4-(10-phenylanthracen-9-yl)phenyl)anthracen-9-yl)-1H-naphtho[2,3-d]imidazole.

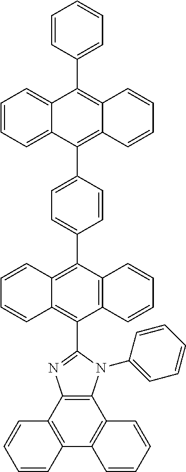 [SMILES: c1ccc(-c2c3ccccc3c(-c3ccc(-c4c5ccccc5c(-c5nc6c7ccccc7c7ccccc7c6n5-c5ccccc5)c5ccccc45)cc3)c3ccccc23)cc1]

1-phenyl-2-(10-(4-(10-phenylanthracen-9-yl)phenyl)anthracen-9-yl)-1H-phenanthro[9, 10-d]imidazole.

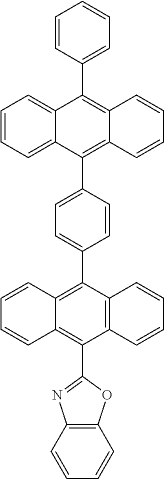 [SMILES: c1ccc(-c2c3ccccc3c(-c3ccc(-c4c5ccccc5c(-c5nc6ccccc6o5)c5ccccc45)cc3)c3ccccc23)cc1]

2-(10-(4-(10-phenylanthracen-9-yl)phenyl)anthracen-9-yl)benzo[d]oxazole.

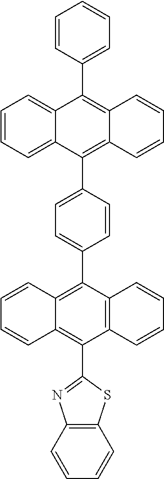 [SMILES: c1ccc(-c2c3ccccc3c(-c3ccc(-c4c5ccccc5c(-c5nc6ccccc6s5)c5ccccc45)cc3)c3ccccc23)cc1]

2-(10-(4-(10-phenylanthracen-9-yl)phenyl)anthracen-9-yl)benzo[d]thiazole.

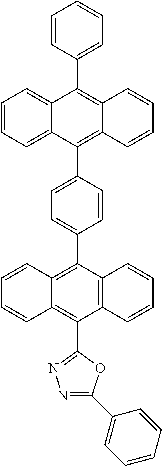 [SMILES: c1ccc(-c2nnc(-c3c4ccccc4c(-c4ccc(-c5c6ccccc6c(-c6ccccc6)c6ccccc56)cc4)c4ccccc34)o2)cc1]

2-phenyl-5-(10-(4-(10-phenylanthracen-9-yl)phenyl)anthracen-9-yl)-1,3,4-oxadiazole.

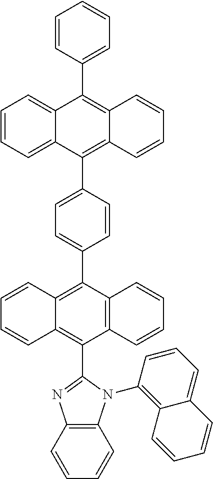 [SMILES: c1ccc(-c2c3ccccc3c(-c3ccc(-c4c5ccccc5c(-c5nc6ccccc6n5-c5cccc6ccccc56)c5ccccc45)cc3)c3ccccc23)cc1]

1-(naphthalen-1-yl)-2-(10-(4-(10-phenylanthracen-9-yl)phenyl)anthracen-9-yl)-1H-benzo[d]imidazole.

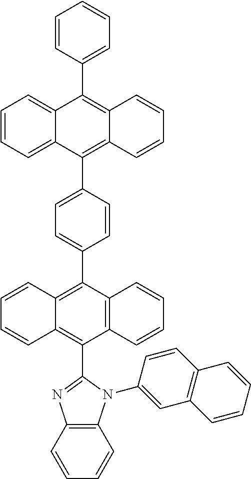 [SMILES: c1ccc(-c2c3ccccc3c(-c3ccc(-c4c5ccccc5c(-c5nc6ccccc6n5-c5ccc6ccccc6c5)c5ccccc45)cc3)c3ccccc23)cc1]

1-(naphthalen-2-yl)-2-(10-(4-(10-phenylanthracen-9-yl)phenyl)anthracen-9-yl)-1H-benzo[d]imidazole.

The dianthracenebenzimidazoles having above structure were chemically synthesized, and identified by a nuclear magnetic resonance spectrometer and mass spectrometer to obtain the results: ¹H NMR (400 MHz, d-DCM) δ8.05(d, J=7.6 Hz, 1H), 8.00-7.97(m, 4H), 7.78-7.72(m, 7H), 7.70-7.65(m, 3H), 7.63-7.59(m, 1H), 7.55-7.47(m, 11H), 7.44-7.40(m, 2H), 7.28-7.26(m, 2H), 7.22-7.19(m, 2H); ¹³C NMR (100 MHz, d-DCM) δ151.32, 139.46, 139.02, 137.96, 137.83, 137.20, 136.57, 136.35, 131.90, 131.86, 131.84, 131.76, 131.75, 131.71, 131.62, 131.52, 130.44, 130.42, 130.17, 129.70, 128.91, 128.89, 128.83, 128.79, 128.62, 128.21, 128.17, 128.00, 127.94, 127.83, 127.64, 127.44, 127.35, 127.10, 126.54, 126.11, 125.99, 125.92, 125.68, 125.56, 124.10, 123.91, 123.86, 123.52, 120.36, 111.24 HRMS (MALDI) m/z calcd for C₅₃H₃₄N₂ 698.2722. obsd. 699.2814.(M⁺).

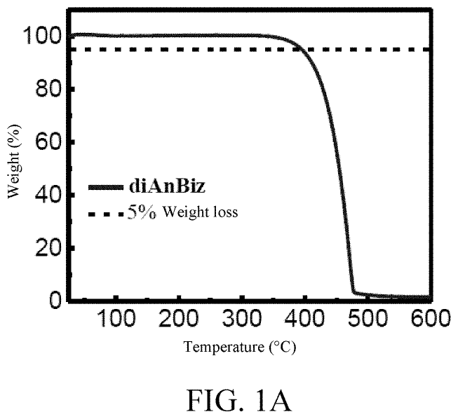

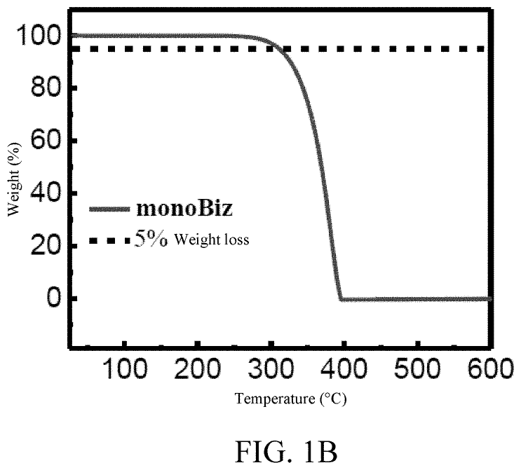

The dianthracenebenzimidazole (diAnBiz) compounds and anthracene-free benzimidazole compounds (monoBiz) were subjected to thermal properties measurement. The conditions for measurement of thermal properties were as follows: a Q20 differential scanning calorimeter (DSC) from TA was used for measuring the glass transition temperature (Tg) and melting point of the compounds. Measurement conditions were as follows: it was repeated twice that in a nitrogen flow of 20 mL/min, the compounds were heated to 350-400° C. at a heating rate of 10° C./min and kept at 400° C. for 1 minute, and then cooled to 30° C. at a cooling rate of 10° C./min, and the result of the second measurement was regarded as the glass transition temperature of the compounds; a Perkin-Elmer 7 thermogravimetric analyzer (TGA) was used for measuring the thermal decomposition temperature of the compounds. Measurement conditions were as follows: in a nitrogen flow, the compounds are heated from room temperature to 800° C. at a heating rate of 10° C./min, when the loss ratio of the compounds under measurement reaches 5 wt %, the temperature was regarded as the thermal decomposition temperature of the compounds. The thermal properties measurement results were shown in Table 1 and FIGS. 1A and 1B.

TABLE 1

| | | Tₘ | T_{d} | T_{g} | g = T_{g}/Tₘ |
|---|---|---|---|---|---|
| Compounds | M.W. | (° C.) | (° C.) | (° C.) | (K/K) |
| diAnBiz | 698.87 | 339 | 395 | 185 | 0.75 |
| monoBiz | 446.55 | 234 | 310 | 108 | 0.75 |

According to Table 1, the thermal decomposition temperature of the diAnBiz compounds is close to 400° C., because the diAnBiz compounds composed of aromatic nuclei are structurally rigid and therefore, when heated, tend to resist high temperature thermal decomposition. Furthermore, the glass transition temperature reaches 185° C., and the thermal stability is high. According to the above mentioned, the anthracene materials of the diAnBiz compounds, for example, can have a good thermal stability and a high triplet energy level, and therefore are ideal host materials of the light emitting layer in an organic light emitting diode.

The dianthracenebenzimidazole (diAnBiz) compounds, anthracene-free benzimidazole compounds (monoBiz), and diphenylanthracene (DPA) are subjected to electrochemical properties measurement. More specifically, an electrochemical analyzer (CH Instruments, CHI 1405, USA) was used for measuring the energy of the highest occupied molecular orbital (EHOMO) and energy of the lowest unoccupied molecular orbital (ELUMO) by cyclic voltammetry and differential-pulse voltammetry (DPV). The oxidation potential measurement conditions were as follows:

Solvent: dichloromethane;

Working electrode: platinum electrode;

Reference electrode: silver/silver chloride;

Auxiliary electrode: platinum wire;

Electrolyte: tetrabutylammonium perchlorate (10⁻¹M);

Scanning speed: 50 mV/sec;

Reduction potential measurement conditions are as follows:

Solvent: N,N-dimethylformamide (DMF) anhydrous;

Working electrode: glassy carbon electrode;

Concentration of the solution to be measured: 10⁻³ M;

standard substance: ferrocene with a concentration of 10⁻³ M.

Furthermore, a potential measured by cyclic voltammetry is not the absolute potential of material, so it is a must that a known substance, commonly ferrocene, should be used as a standard substance; according to the difference between measured potential and that of the standard substance, the ELUMO, EHOMO of the material can be estimated according to the following formula³⁸:

E_{HOMO}=−1.2×(E_{DPV}^{ox}−E^{Fc+/Fc})+(−4.8) eV

E_{LUMO}=−0.92×(E_{DPV}^{re}−E^{Fc+/Fc})+(−4.8) eV

Wherein E_{DPV}^{ox} is the first oxidation peak in the DPV graph, E_{DPV}^{re} is the first reduction peak in the DPV graph, and E^{Fc+/Fc} is calculated by the total of the Eₚₐ and E_{pc} of the ferrocene in the CN graph and dividing the total by 2. Since a material-solution state energy level can be obtained by the experiment, it can be initially judged whether the material has an energy level alignment in elements, and further can be confirmed by measure the state energy levels of a film state.

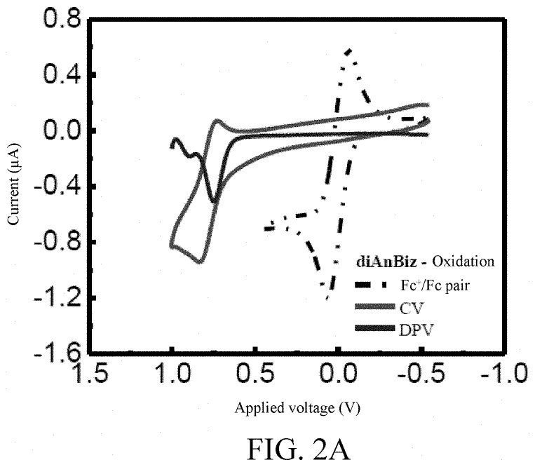

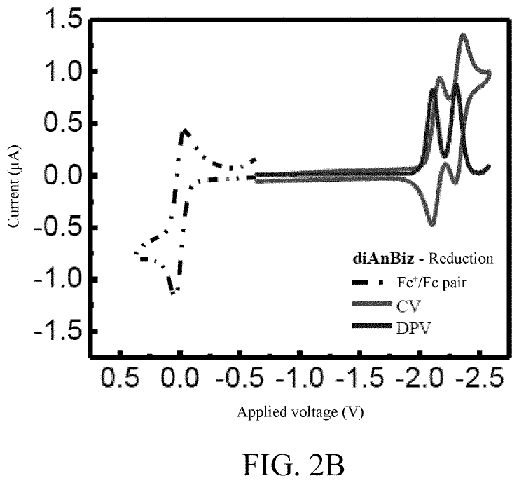

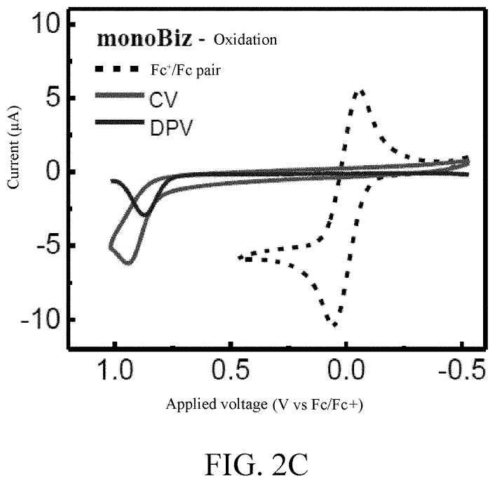

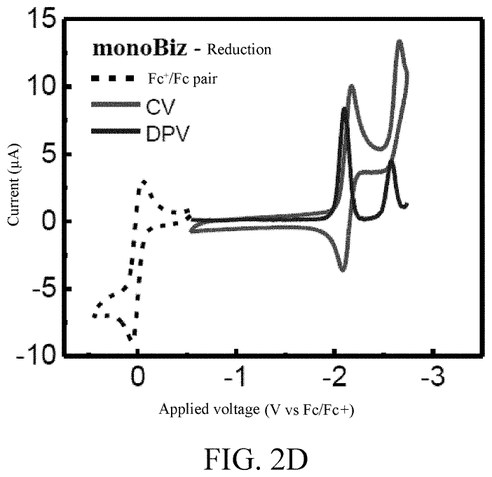

The results from measurement on said electrochemical properties are shown in Table 2, and FIGS. 2A-2D.

TABLE 2

| | E_{DPV}^{ox} | E_{DPV}^{re} | E_{HOMO}/E_{LUMO} | E_{g}^{sol} |
|---|---|---|---|---|
| Compounds | (V) | (V) | (eV) | (eV) |
| diAnBiz | 0.75 | −2.10 | −5.70/−2.87 | 2.83 |
| monoBiz | 0.87 | −2.10 | −5.84/−2.87 | 2.97 |
| DPA | 0.73 | −2.11 | −5.68/−2.86 | 2.82 |

According to Table 2, the energy level of anthracene materials can be obtained to select a more suitable host material, electron or hole blocking layer, and electron or hole transport layer.

The diAnBiz and monoBiz were subjected to measurement on the photophysical properties. Photophysical properties measurement were under conditions as follows: diAnBiz and monoBiz solutions with a concentration of 10⁻⁵M were obtained with spectroscopic grade tetrahydrofuran (THF) as solvent, and were subjected to ultraviolet-visible (UV) absorption spectroscopy and normal temperature fluorescence (FL) emission spectroscopy respectively; diAnBiz and monoBiz solutions with a concentration of 10⁻⁵M were obtained with spectroscopic grade 2-methyltetrahydrofuran as solvent, and were subjected, in the presence of liquid nitrogen as a refrigerant and at a low temperature of 77K, to low temperature phosphorescence (PH) emission spectroscopy and low temperature fluorescence (LTFL) emission spectroscopy respectively (a Shimadzu UV-1601PC uv/visible spectrophotometer and a Hitachi F-4500 are used). The obtained spectroscopic data were normalized.

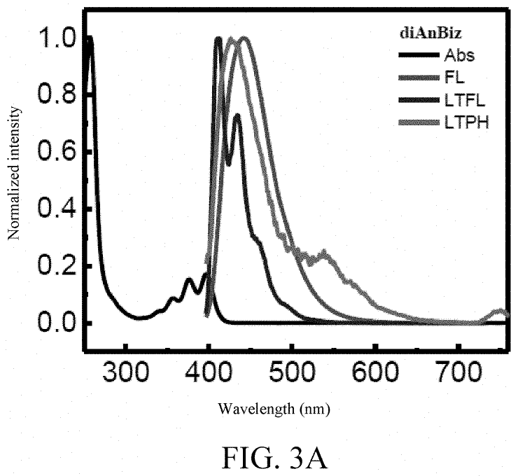

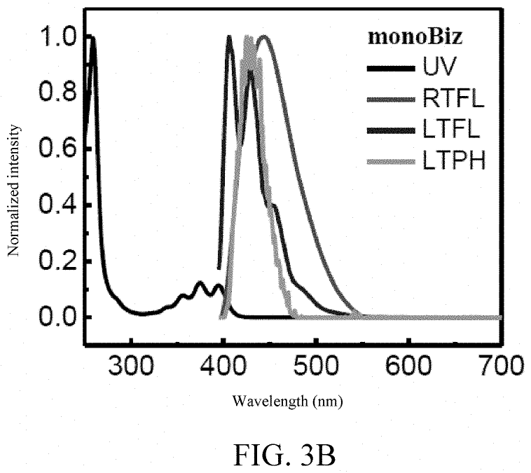

The results from on measurement photophysical properties are shown in Table 3 and FIGS. 3A and 3B.

TABLE 3

| | ^{a}λₘₐₓ^{Abs} | ^{b}λₘₐₓ^{FL} | ^{c}λₘₐₓ^{LTFL} | ^{d}λₒₙₛₑₜ^{Abs} | ^{e}E_{g}^{sol} | |
|---|---|---|---|---|---|---|
| Compounds | (nm) | (nm) | (nm) | (nm) | (eV) | ^{f}PLQY |
| diAnBiz | 397 | 442 | 412 | 418 | 2.97 | 0.91 |
| monoBiz | 375 | 444 | 406 | 412 | 3.01 | 0.88 |

wherein

Abs: absorption;

FL: fluorescence;

LTFL: low temperature fluorescence;

LTPH: low temperature phosphorescence;

^{a}: the maximum uv-visible absorption wavelength of the compound;

^{b}: the maximum fluorescence emission wavelength of the compound at room temperature;

^{c}: the maximum fluorescence emission wavelength at a temperature of 77K;

^{d}: the initial uv-visible absorption wavelength of the compound;

^{e}E_{g}^{sol}=1240.8/λₒₙₛₑₜ^{Abs} (nm);

^{f}: quantum yield Q=Q_{R}×(I/I_{R})×(OD_{R}/OD)×(n/n_{R})²; all measurements were carried out in toluene.

According to Table 3, for example, the singlet energy level of the anthracene materials of the diAnBiz compounds can be calculated by the listed formula, and a more suitable host material is selected. Furthermore, diAnBiz emits blue fluorescence at 442 nm according to fluorescence measurement.

On the other hand, according to the overlap between LTPH and LTFL in FIG. 3, it can be judged that diAnBiz is capable of triplet-triplet annihilation upconversion (TTA-UC), and can emit fluorescence by converting a triplet exciton into a singlet exciton by TTA-UC, so in LTPH measurement, fluorescence is actually measured.

Furthermore, the conditions for TTA-UC tests on diAnBiz were as follows:

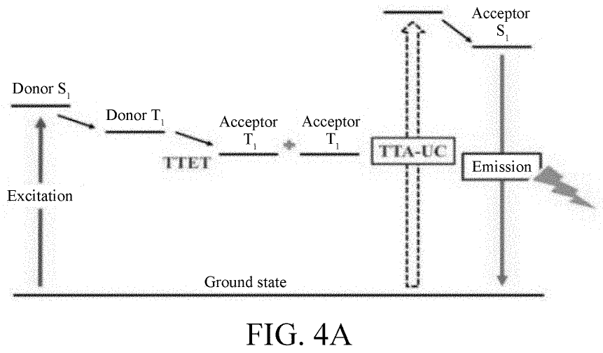

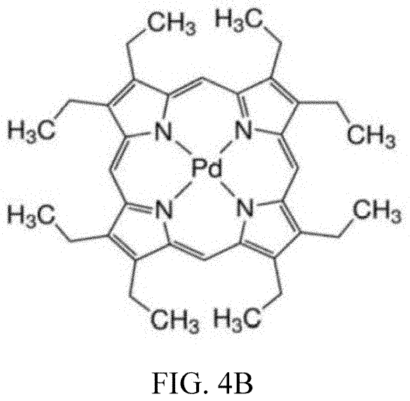

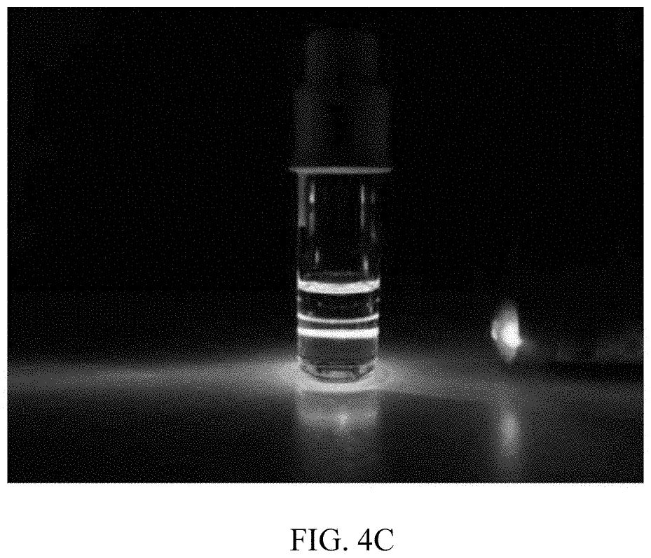

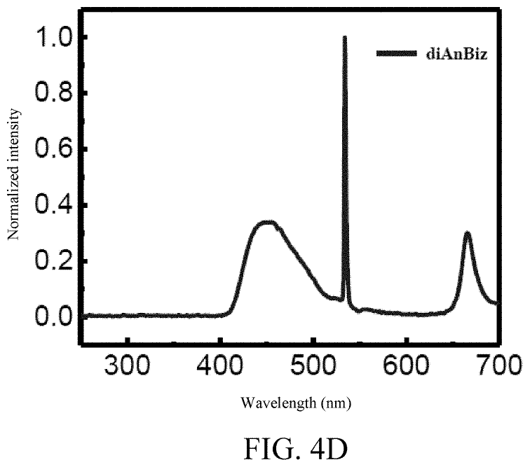

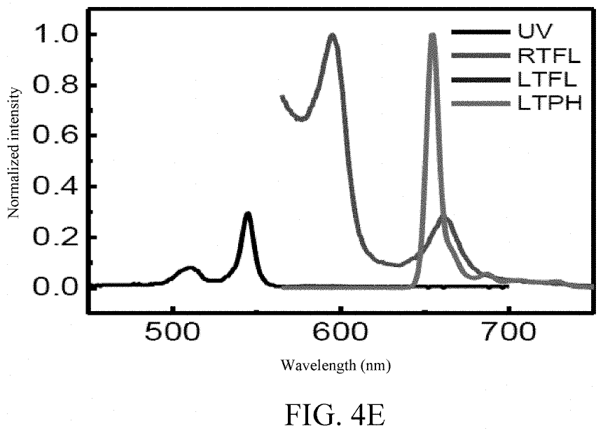

sensitizer: 2,3,7,8,12,13,17,18-Octaethyl-21H,23H-porphine palladium(II) (PdOEP) with a concentration of 10⁻⁵ M. According to the TTA-UC mechanism shown in FIG. 4A, PdOEP serves as a triplet exciton donor. The structure of PdOEP is shown in FIG. 4B.

DiAnBiz compound: acceptor with a concentration of 10⁻⁴ M.

Solvent: xylenes.

Excitation light source: green laser pen (λₑₓ=532±10 nm).

The solution is deoxidized by Ar_{(g)}, a singlet excited state is produced by green light excitation of the sensitizer, the sensitizer containing Pd can quickly perform intersystem crossing to a triplet excited state, triplet-triple energy is transferred to the triplet of the compound, and finally fluorescence is emitted by the TTA-UC of an exciton to a singlet excited state at a higher energy level. Therefore, green light with a longer wavelength can be used for producing blue light with a shorter wavelength.

More specifically, PdOEP, having a heavy atom effect, can subject absorbed energy to quick intersystem crossing from a singlet (S₁) to a triple (T₁). And, energy is transferred by triplet triplet energy transfer (TTET) to a triplet exciton acceptor (namely, diAnBiz). At this point, if diAnBiz is capable of TTA-UC, two triple excitons can produce a singlet exciton and another diAnBiz returns to a ground state (S₀) according to the formula T₁+T₁−>S₁+S₀.

According to the embodiment of FIG. 4C, the diAnBiz solution, when excited by a green laser pen, emits blue light. According to the embodiment of FIG. 4D, the analytical data of a fluorescence emission spectrometer is shown in FIG. 4D. Wherein, the wave at 534 nm is produced by the green laser pen, and the waveform at 449 nm is the blue light emitted by the TTA-UC of diAnBiz. According to the photophysical properties measurement results of PdOEP shown in FIG. 4E, it can be verified that the LTPH of PdOEP exists at 665 nm.

In an embodiment of the invention, a dianthracenebenzimidazole compound of anthracene materials can be prepared according to the following equation.

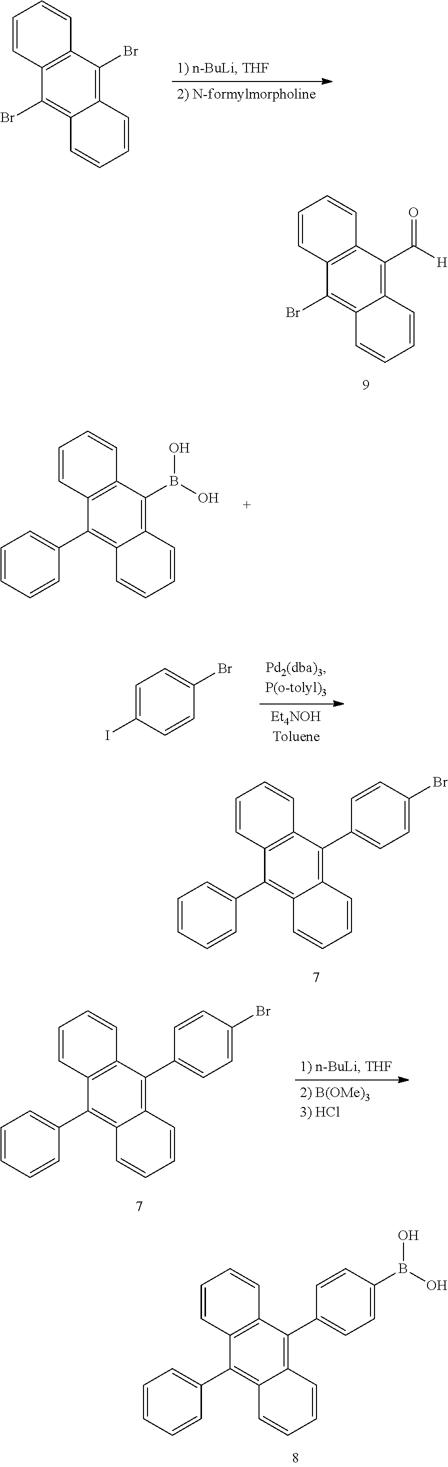

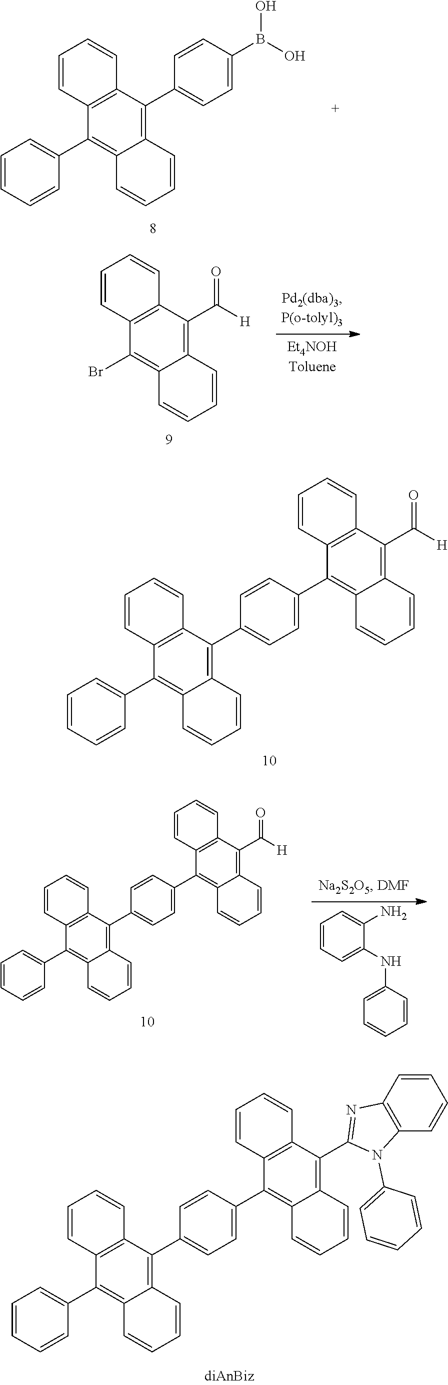

More specifically, in the equation, the anthracene compound 9, 10-bromoanthracene-9-carbaldehyde, has the following structure:

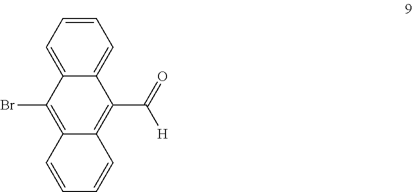 [SMILES: O=Cc1c2ccccc2c(Br)c2ccccc12]

In an embodiment, the preparation method comprises: placing 9,10-dibromoanthracene (3.36 g, 10 mmol) in a 100 ml two-neck bottle with a stir bar, adding anhydrous tetrahydrofuran (THF, 40 ml) after carrying out argon replacement thrice, and placing the bottle in a dry ice-acetone bath; adding n-butyllithium (1.6 M, 6.3 ml, 10.1 mmol) after the temperature balances, continuously stirring the mixture for 1 hour, and adding molecular sieve dried N-formylmorpholine (1.02 ml, 10.1 mmol); after the temperature cools to room temperature, continuously stirring the mixture for 4 hours, adding an HCL aqueous solution (1 M, 2 ml), and removing THF by vortex concentration; dissolving the mixture in dichloromethane, and washing with deionized water and a saturated salt solution once in turn; drying the organic layer with anhydrous magnesium sulfate, and subjecting to silica gel column chromatography (eluent: HEX : DCM=2 : 1); after concentration, recrystallizing the solid with normal hexane and dichloromethane to obtain 1.3 g of a bright yellow needle crystal, whose yield reaches 46%.

The structural identification data is as follows: ¹H NMR (400 MHz, CDCl₃) δ 11.50 (s, 1H), 8.9-8.88 (m, 2H), 8.88-8.67(m, 2H), 7.73-7.65(m, 4H) ; ¹³C NMR (100 MHz, CDCl₃) δ 193.30, 131.94, 130.30, 129.01, 128.91, 127.40, 123.84.

The anthracene compound 7, 9-(4-bromophenyl)-10-phenylanthracene, has the structure:

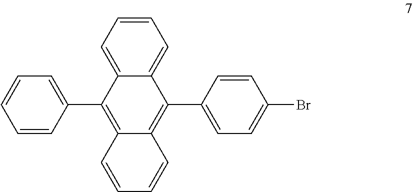 [SMILES: Brc1ccc(-c2c3ccccc3c(-c3ccccc3)c3ccccc23)cc1]

In an embodiment, the preparation method comprises: placing (10-phenylanthracen-9-yl)boronic acid (2.98 g, 10 mmol), 1-bromo-4-iodobenzene (8.49 g, mmol), tris(dibenzylideneacetone)dipalladium(0) (0.915 g, 0.1 mmol), tri(o-tolyl)phosphine (0.913 g, 0.15 mmol) in a 250 ml two-neck bottle with a stir bar, and mixing with deoxidized toluene (45 ml) and a 20% tetramethylammonium hydroxide aqueous solution (45 ml) after carrying out argon replacement thrice; heating the mixture to 115° C., and refluxing for 18 hours; after the reaction, subjecting the mixture to vortex concentration to remove toluene, and carrying out extraction twice with dichloromethane; washing the organic layer with a saturated salt solution once, dehydrating with anhydrous magnesium sulfate, and subjecting to silica gel column chromatography (eluent: Hex: DCM=10: 1); after concentration, recrystallizing the solid with normal hexane and dichloromethane to obtain 3.01 g of a white solid, whose yield reaches 73%.

The structural identification data is as follows: ¹H NMR (400 MHz, d-DMSO) δ 7.86(d, J=8.4 Hz), 7.69-7.56(m, 7H), 7.47-7.43(m, 8H) ; ¹³C NMR (100 MHz, CDCl₃) δ 166.12, 163.31, 142.12, 138.13, 134.84, 132.92, 131.28, 130.89, 129.84, 129.21, 128.90, 128.52, 128.09, 128.01, 127.97, 127.66, 127.53, 127.38, 127.16, 125.60, 125.11, 123.33, 121.19, 117.49 HRMS (MALDI) m/z calcd for C₂₆H₁₇Br 408.0514. obsd. 408.0522.

The anthracene compound 8, (4-(10-phenylanthracen-9-yl)phenyl)boronic acid, has the structure:

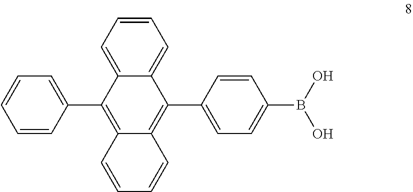 [SMILES: OB(O)c1ccc(-c2c3ccccc3c(-c3ccccc3)c3ccccc23)cc1]

In an embodiment, the preparation method comprises: placing the compound 7 (2.00 g, 4.88 mmol) in a 100 ml two-neck bottle with a stir bar, adding dried tetrahydrofuran (THF, 30 ml) after carrying out argon replacement thrice, and placing the bottle in a dry ice-acetone bath; adding n-butyllithium (1.6 M, 3.4 ml, 5.44 mmol) after the temperature balances, continuously stirring the mixture for 1 hour, adding trimethyl borate (1.30 ml, 11.64 mmol), removing the bath, and continuously stirring the mixture for 24 hours; adding an HCL aqueous solution (1 M, 20 ml), stirring the mixture for 1 hour, removing the tetrahydrofuran by vortex concentration, and carrying out extraction twice with ethyl acetate; washing the organic layer with water and a saturated salt solution once in turn, dehydrating with anhydrous magnesium sulfate, and finally recrystallizing with ethyl acetate/normal hexane to obtain 1.03 g of the compound 8, whose yield reaches 56%.

The structural identification and synthetic method reference the following: Moorthy, J. N.; Venkatakrishnan, P.; Natarajan, P.; Huang, D.-F.; Chow, T. J., De Novo Design for Functional Amorphous Materials: Synthesis and Thermal and Light-Emitting Properties of Twisted Anthracene-Functionalized Bimesitylenes. Journal of the American Chemical Society 2008, 130 (51), 17320-17333.

The anthracene compound 10, 10-(4-(10-phenylanthracen-9-yl)phenyl) anthracene-9-carbaldehyde, has the structure:

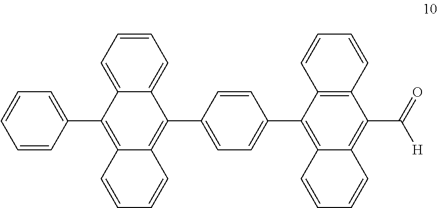 [SMILES: O=Cc1c2ccccc2c(-c2ccc(-c3c4ccccc4c(-c4ccccc4)c4ccccc34)cc2)c2ccccc12]

In an embodiment, the preparation method comprises: placing the compound 8 (1 g, 2.67 mmol), the compound 9 (0.693 g, 2.43 mmol), tris(dibenzylideneacetone)dipalladium(0) (0.223 g,0.24 mmol), tri(o-tolyl)phosphine (0.221 mg,0.73 mmol) in a 150 ml two-neck bottle with a stir bar, mixing with deoxidized toluene (20 ml) and a 20% tetramethylammonium hydroxide aqueous solution (20 ml) after carrying out argon replacement thrice, heating to 115° C., and refluxing for 18 hours; after the reaction, subjecting the mixture to vortex concentration to remove toluene, and carrying out extraction twice with dichloromethane; washing the organic layer with a saturated salt solution once, dehydrating with anhydrous magnesium sulfate, and subjecting to silica gel column chromatography (eluent: pure toluene); after concentration, obtaining 0.7 g of a yellow solid, whose yield reaches 54%.

The structural identification data is as follows: ¹ NMR (400 MHz, d-DMSO) δ 11.60(s, 1H), 9.12(d, J=8.8 Hz, 2H), 7.95(d, J=8.8 Hz, 2H), 7.89(d,J=8.8 Hz, 2H), 7.85-7.81(m, 2H), 7.78-7.76(m, 2H), 7.73-7.68(m, 6H),7.66-7.64(m, 3H), 7.60-7.57(m, 2H), 7.53-7.49(m, 4H); ¹³C NMR (100 MHz, d-DCM) δ 194.04, 145.77, 145.16, 141.00, 140.82, 139.57, 139.50, 138.91, 138.09, 138.04, 137.16, 133.50, 132.23, 132.15, 132.06, 131.87, 131.42, 131.33, 130.69, 130.57, 129.21, 129.06, 129.00, 128.69, 128.49, 128.41, 128.33, 128.27, 128.17, 128.09, 127.63, 127.44, 126.37, 126.31, 126.04, 125.90, 125.74, 124.16, 124.10, 123.97 HRMS (MALDI) m/z calcd for C₄₁H₂₆O 534.1983. obsd.534.1958.

An anthracene compound, 1-phenyl-2-(10-(4-(10-phenylanthracen-9-yl) phenyl)anthracen-9-yl)-1H-benzo[d]imidazole (dianthracenebenzimidazole), has the structure:

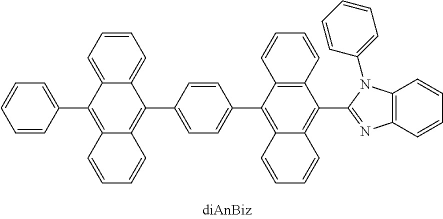 [SMILES: c1ccc(-c2c3ccccc3c(-c3ccc(-c4c5ccccc5c(-c5nc6ccccc6n5-c5ccccc5)c5ccccc45)cc3)c3ccccc23)cc1]

In an embodiment, the preparation method comprises: placing the compound 10 (0.84 g, 1.57 mmol), N-phenyl-1,2-benzenediamine (0.3 g, 1.62 mmol), and sodium metabisulfite (1.07 g, 5.61 mmol) in a 50 ml one-neck bottle with a stir bar, installing a reflux unit and a three-way valve, and carrying out argon replacement thrice; mixing with dehydrated N,N-dimethylformamide (DMF, 10 ml) and carrying out a reaction in a microwave reactor (reaction conditions: heating the mixture to 130° C. within 1 minute, keeping at 130° C. at a power of 150 W, and stirring for 3 hours); after the reaction, obtaining an orange precipitate by dripping the product in quickly stirred deionized water (200 ml), subjecting to suction filtration, washing with deionized water, and subjecting to silica gel column chromatography (eluent: Toluene:EA=15:1); after vortex concentration, subjecting the precipitate to thermal washing with acetone for 3 hours, and subjecting to suction filtration and continuously washing with acetone to obtain about 0.7 g of a yellowish solid, whose yield reaches 64%.

The structural identification data is as follows: ¹H NMR (400 MHz, d-DCM) δ8.05(d, J=7.6 Hz, 1H), 8.00-7.97(m, 4H), 7.78-7.72(m, 7H), 7.70-7.65(m, 3H), 7.63-7.59(m, 1H), 7.55-7.47(m, 11H), 7.44-7.40(m, 2H), 7.28-7.26(m, 2H), 7.22-7.19(m, 2H) ; ¹³C NMR (100 MHz, d-DCM) δ 151.32, 139.46, 139.02, 137.96, 137.83, 137.20, 136.57, 136.35, 131.90, 131.86, 131.84, 131.76, 131.75, 131.71, 131.62, 131.52, 130.44, 130.42, 130.17, 129.70, 128.91, 128.89, 128.83, 128.79, 128.62, 128.21, 128.17, 128.00, 127.94, 127.83, 127.64, 127.44, 127.35, 127.10, 126.54, 126.11, 125.99, 125.92, 125.68, 125.56, 124.10, 123.91, 123.86, 123.52, 120.36, 111.24 HRMS (MALDI) m/z calcd for C53H34N2 698.2722. obsd.699.2814.(M⁺).

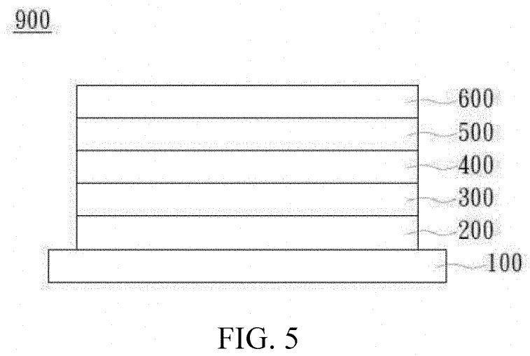

In an embodiment show in FIG. 5, the organic light emitting diodes 900 include a substrate 100, a first conducting layer 200, a hole transport layer 300, a light emitting layer 400, an electron transport layer 500, and a second conducting layer 600. The first conducting layer 200 is disposed on the substrate 100. The hole transport layer 300 is disposed on the first conducting layer 200. The light emitting layer 400 is disposed on the hole transport layer 300, and has the anthracene materials with the structure of the following formula (1):

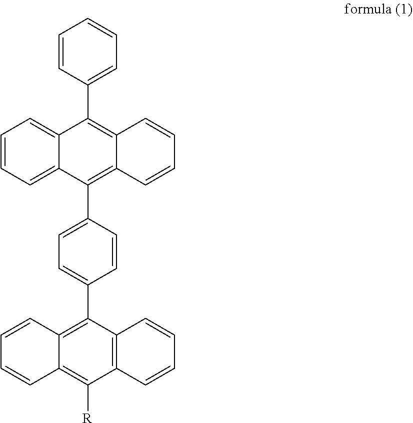

wherein, R is selected from the group consisting of the following groups:

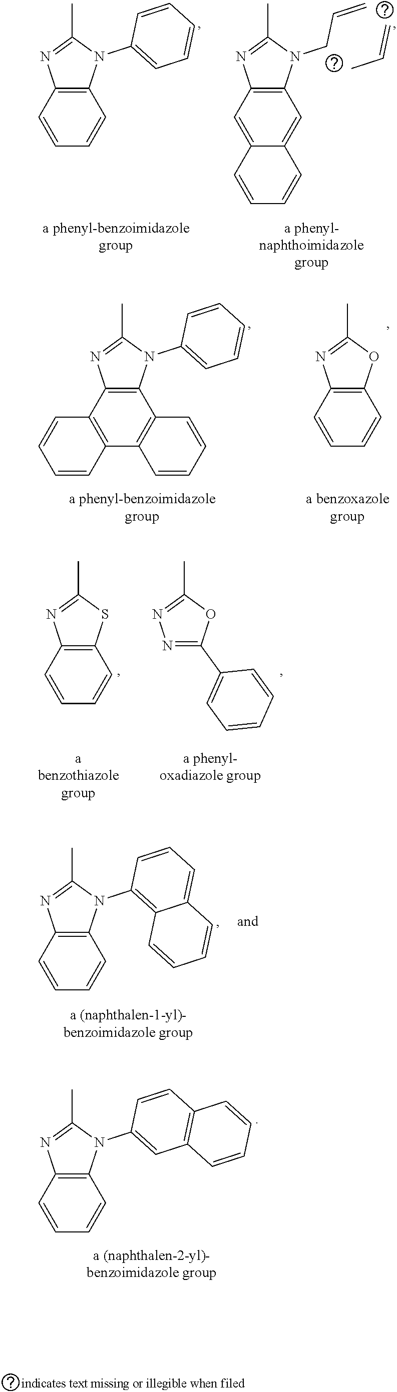

The electron transport layer 500 is disposed on the light emitting layer 400. The second conducting layer 600 is disposed on the electron transport layer 500.

In an embodiment of the invention, the substrate 100 can be a glass substrate or a plastic substrate. Wherein, the substrate 100 can have a certain transparency, and further can be transparent. In an embodiment of the invention, the first conducting layer 200 is an anode preferably with a working function greater than 4.5 eV. The first conducting layer 200 can be made from an indium tin oxide (ITO), tin oxide, gold, silver, platinum, or copper. The hole transport layer 300, without special material limits, can be made from common material compounds, including triaromatic amine derivatives such as TAPC (4,4′-Cyclohexylidenebis[N,N-bis(4-methylphenyl)benzenamine]), mCP (1,3-Bis(N-carbazolyl)benzene), TPD (N,N′-Bis(3-methylphenyl)-N,N′-diphenylbenzidine), or NPB (α-naphylhenyldiamine).

The electron transport layer 500, also without special material limits, can be made from common material compounds. Examples of common materials for electron transport layers are as follows: DPPS (Diphenylbis(4-(pyridin-3-yl)phenyl)silane), LiF, AlQ₃, Bebq₂ (Bis(10-hydroxybenzo[h]quinolinato)beryllium), TAZ (3 -(Biphenyl-4-yl)-5-(4-tert-butylphenyl)-4-phenyl-4H-1,2,4-triazole) or BCP (2,9-Dimethyl-4,7-diphenyl-1,10-phenanthroline). The first conducting layer 600 is a cathode preferably with a smaller working function. Examples of the materials for the first conducting layer 600 can be indium, aluminum, an indium magnesium alloy, magnalium, an aluminum lithium alloy, or a magnesium silver alloy.

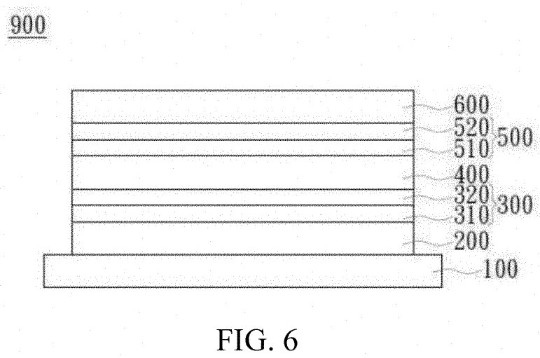

In a different embodiment shown in FIG. 6, the hole transport layer 300 includes an electron injection layer 310 and an hole transfer layer 320 disposed on the electron injection layer 310, and the electron transport layer 500 includes an electron transfer layer 510 and an electron injection layer 520 disposed on the electron transfer layer 510.

In an embodiment, organic light emitting diodes are prepared by thermal evaporation deposition. The configuration is as follows: the first conducting layer ITO/electron injection layer TAPC (500 Å)/hole transfer layer mCP (100 Å)/light emitting layer (host material: light emitting layer) (300 Å)/electron transfer layer DPPS (500 Å)/electron injection layer LiF (0.8 nm)/second conducting layer AI (100 nm). Wherein, the light emitting layer uses diAnBiz as a luminous body material. Namely, organic light emitting diodes are in a film state. Wherein the energy levels of the film state are shown in Table 4.

TABLE 4

| | E_{HOMO} | E_{LUMO} | E_{g} |
|---|---|---|---|
| Compounds | (eV) | (eV) | (eV) |
| diAnBiz | 5.80 | 2.94 | 2.86 |

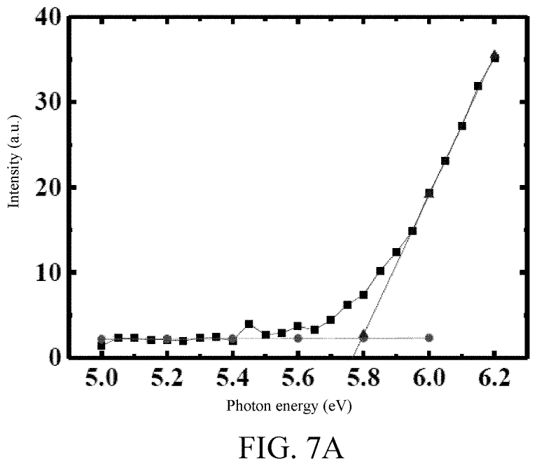

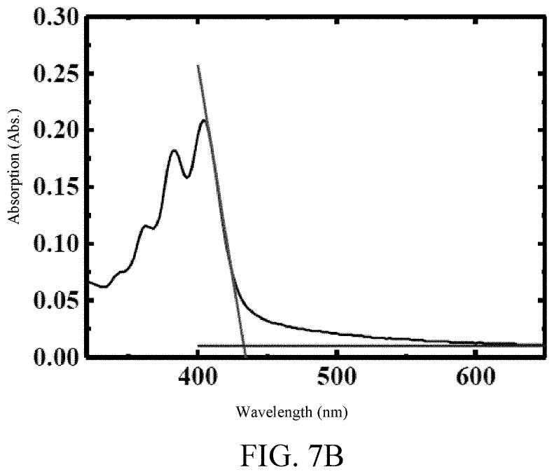

On the other hand, the energy of the highest occupied molecular orbital (EHOMO) of diAnBiz can be obtained according to the onset of FIG. 7A, the onset of FIG. 7B can be obtained according to the formula: Eg =1240.8/λₒₙₛₑₜ^{Abs}(nm), and the energy of the lowest unoccupied molecular orbital (ELUMO) can be obtained according to the formula: E_{g}=E_{HOMO}−E_{LUMO}.

Compared are the elements with the same configuration that are respectively made from diAnBiz, the reference substance monoBiz, and commercially available 9,10-Bis(2-naphthyl)anthrace (ADN). The configuration is as follows: the first conducting layer ITO/electron injection layer TAPC (500 Å)/hole transfer layer mCP (100 Å)/light emitting layer (host material: light emitting layer) (300 Å)/electron transfer layer DPPS (500 Å)/electron injection layer LiF (0.8 nm)/second conducting layer AI (100 nm). Wherein the monoBiz and 9,10-Bis(2-naphthyl)anthrace have the following respective structures:

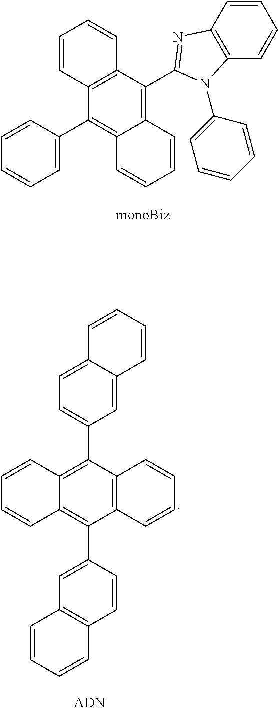

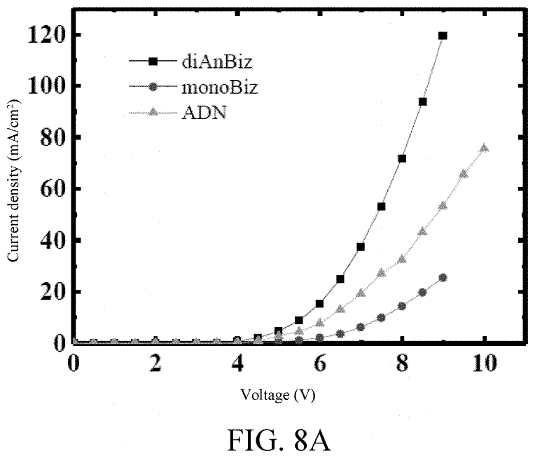

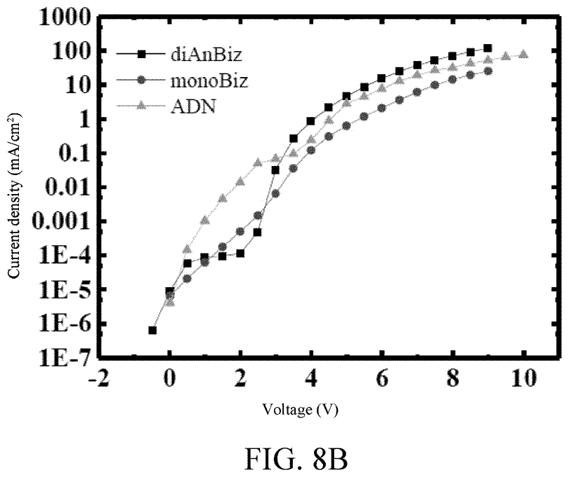

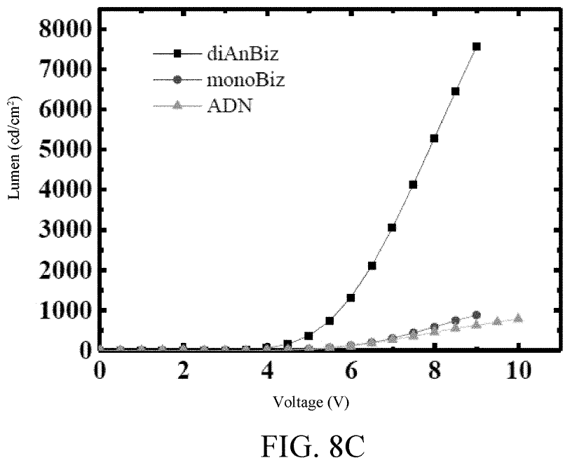

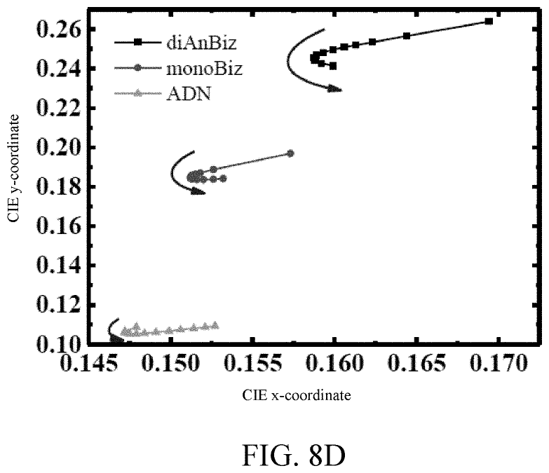

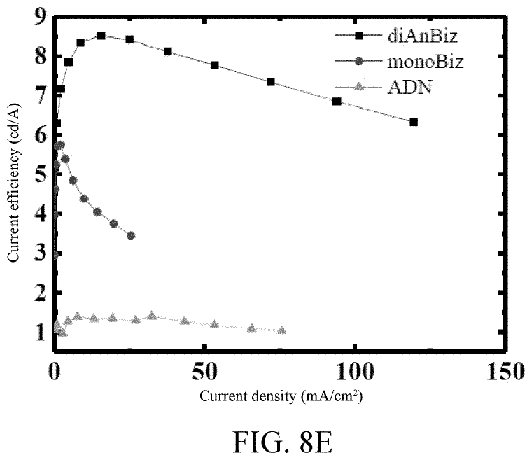

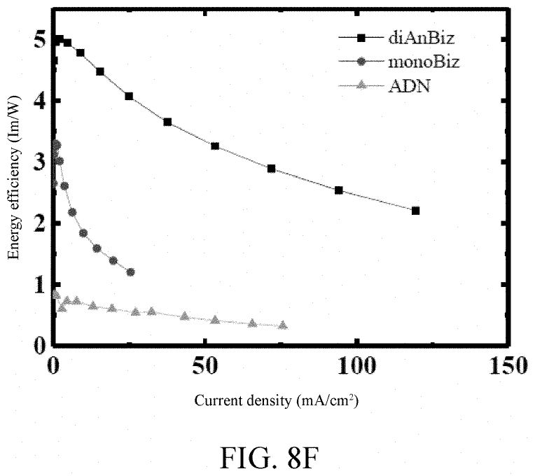

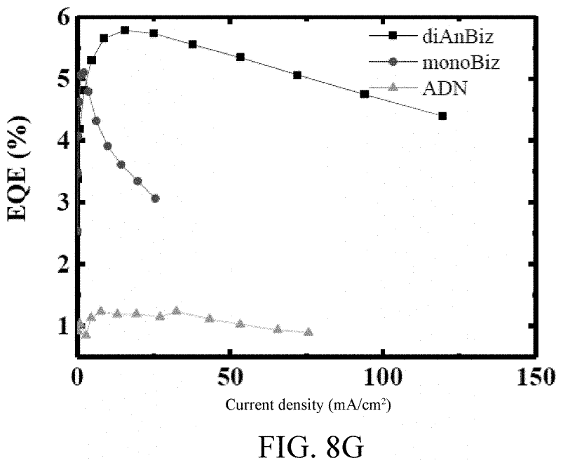

The measurement results are shown in FIGS. 8A-8G and Tables 5-1 and 5-2.

TABLE 5-1

| Compounds used | Voltage (V) @ |
|---|---|
| in the elements | 1 mA/cm² |
| diAnBiz | 4.04 |
| monoBiz | 5.32 |
| ADN | 4.52 |

TABLE 5-2

| Compounds used | max. C.E. | max. P.E. | max. EQE |
|---|---|---|---|
| in the elements | (cd/A) | (lm/W) | (%) |
| diAnBiz | 8.52 | 5.01 | 5.78 |
| monoBiz | 5.75 | 3.30 | 5.09 |
| ADN | 1.40 | 0.83 | 1.23 |

According to the measurement results, it can be found that the maximum luminance, maximal current efficiency (max. C.E.), maximal power efficiency (max. P.E.), and maximal external quantum efficiency (max. EQE) of diAnBiz made elements all are better than those of monoBiz and ADN made elements, and diAnBiz made elements have a starting voltage (4.04 V) that is lower than that of monoBiz and ADN made elements.

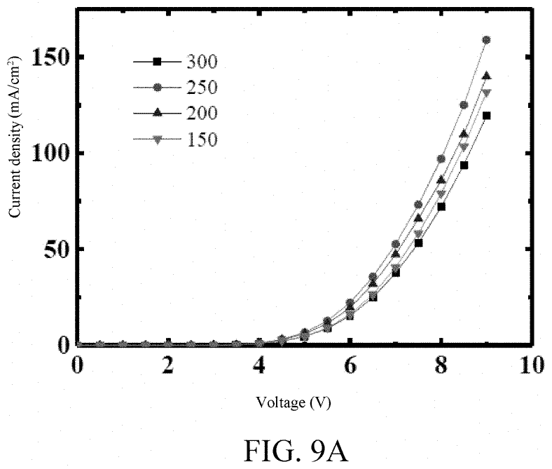

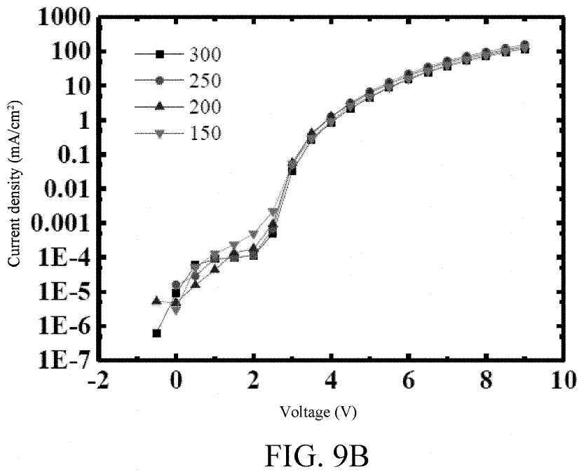

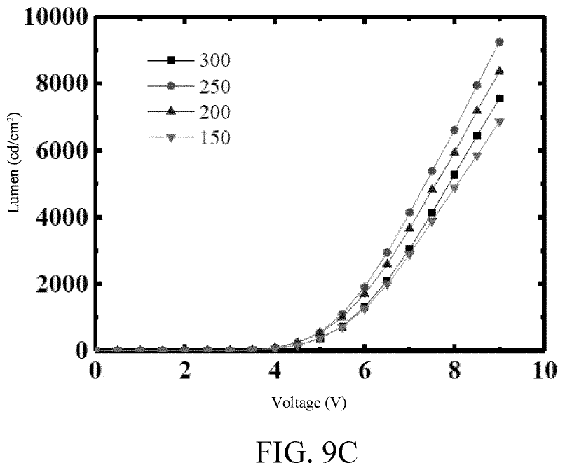

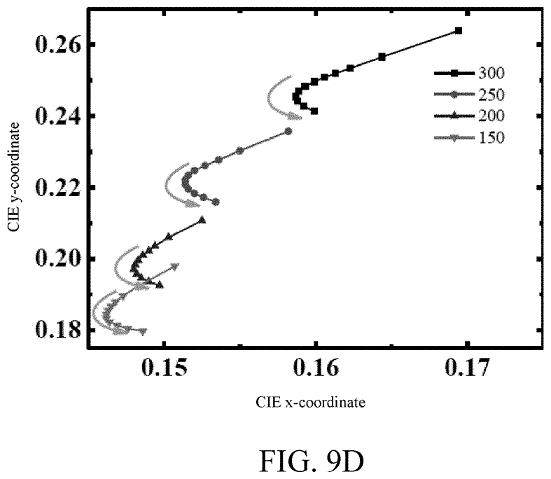

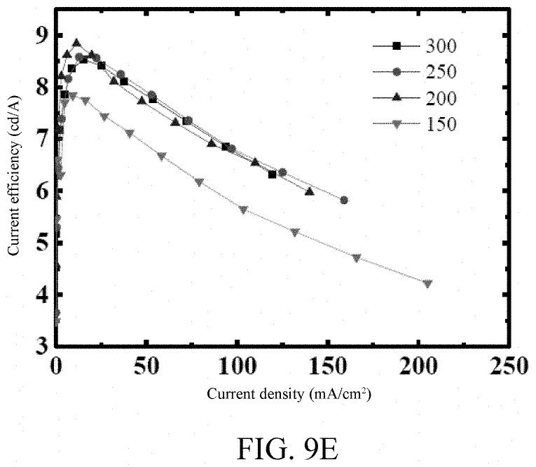

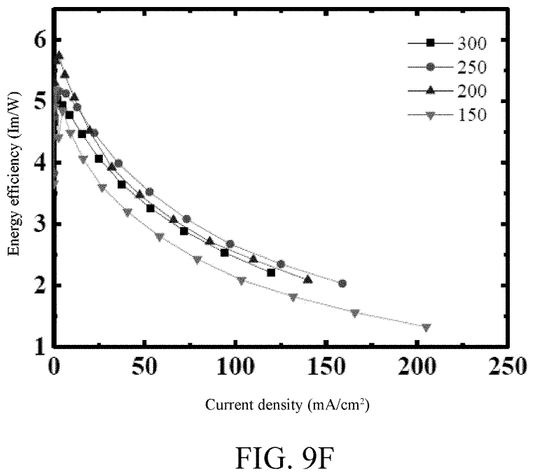

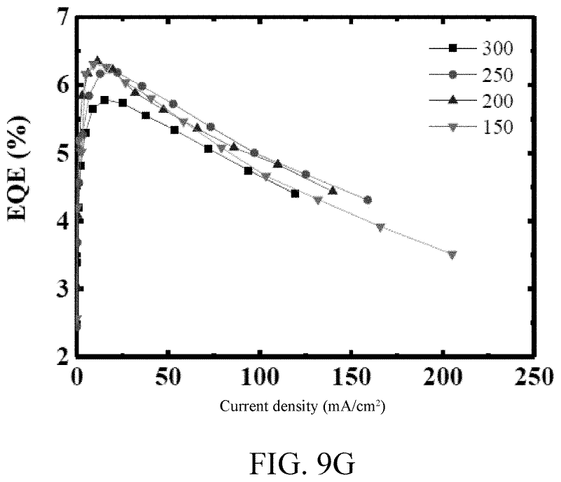

FIGS. 9A-9G and Tables 6-1 and 6-2 show the measurement results of organic light emitting diodes only different in thickness that are made from diAnBiz as host material.

TABLE 6-1

| The thickness of the | Voltage (V) @ |
|---|---|
| light emitting layer (Å) | 1 mA/cm² |
| 300 | 4.04 |
| 250 | 3.86 |
| 200 | 3.85 |
| 150 | 4.02 |

TABLE 6-2

| The thickness | | | |
|---|---|---|---|
| of the light | | | |
| emitting layer | max. C.E. | max. P.E. | max. EQE |
| (Å) | (cd/A) | (l m/W) | (%) |
| 300 | 8.52 | 5.01 | 5.78 |
| 250 | 8.58 | 5.16 | 6.18 |
| 200 | 9.15 | 5.75 | 6.73 |
| 150 | 7.84 | 5.19 | 6.31 |

According to the measurement results, it can be found that the light emitting layer with a thickness of 200 Å has the lowest starting voltage (3.85V), the maximal current efficiency (9.15 cd/A), maximal power efficiency (5.75 lm/W), and maximal external quantum efficiency (6.73%), and therefore under the condition of the thickness of 200 Å of the light emitting layer, further improvements are made.

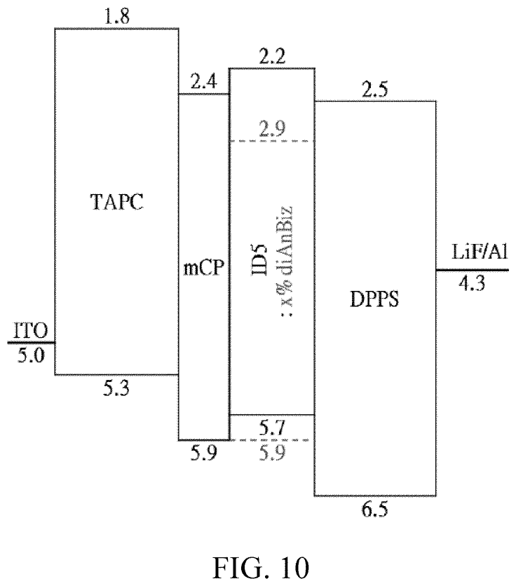

FIG. 10 is an element configuration and energy level diagram.

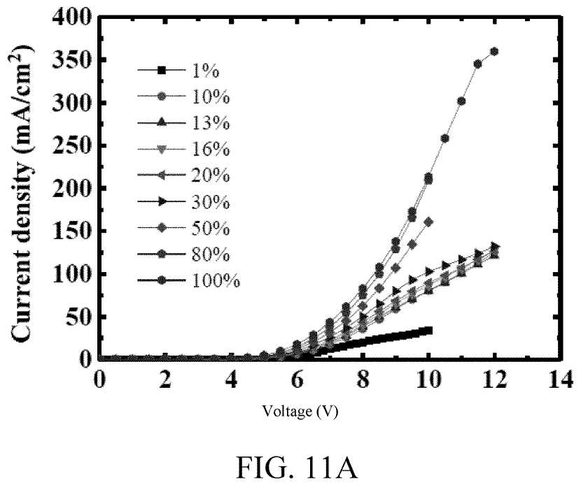

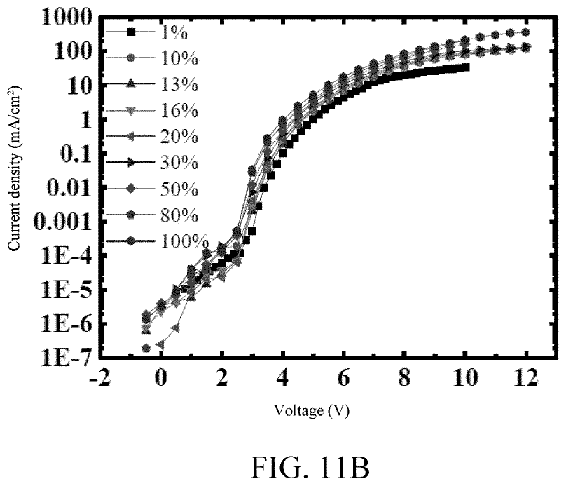

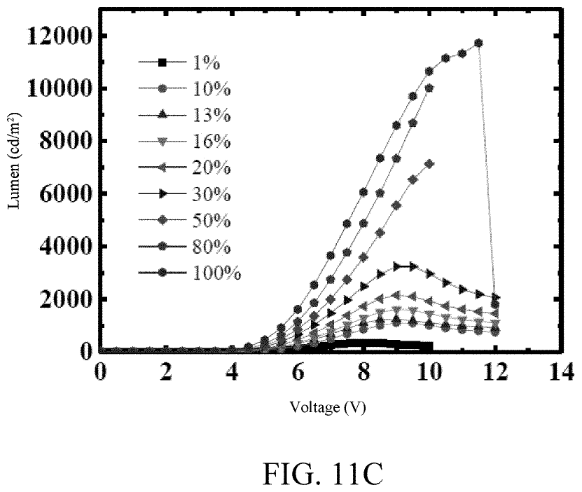

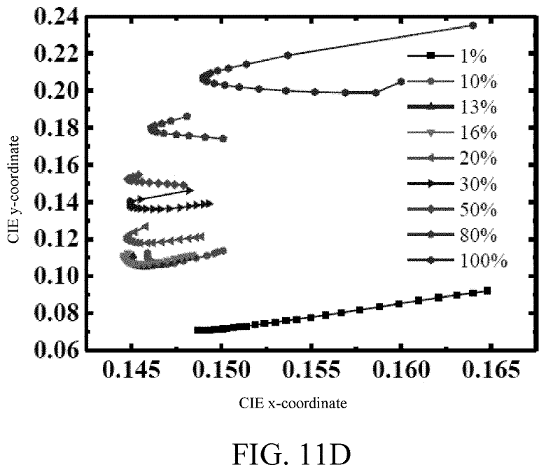

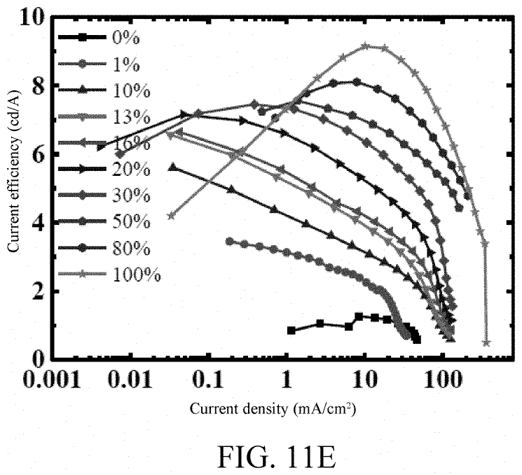

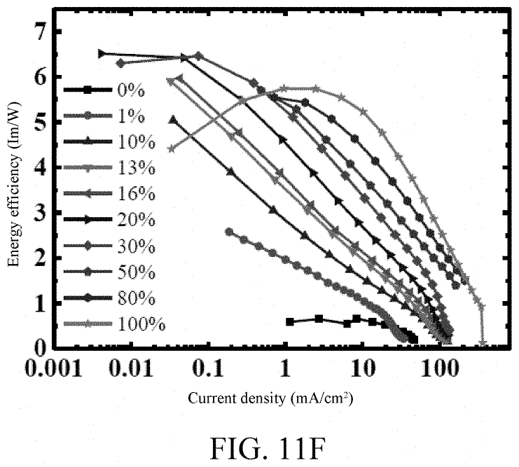

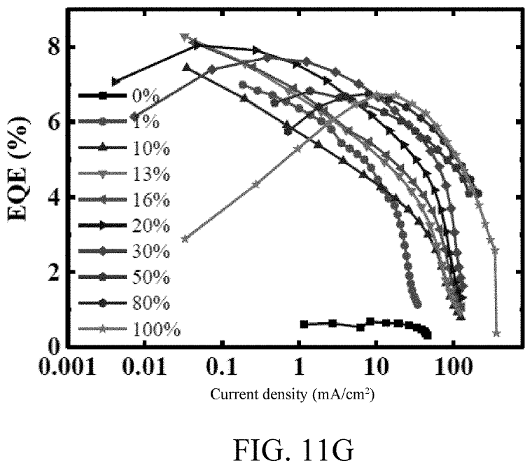

Under the condition of the thickness of 200 Å of the light emitting layer, 9,9′-(2-(1-phenyl-1H-benzo [d]imidazol-2-yl)-1,3 -phenylene)bis(9H-carbazole) (o-DiCbzBz) as a host is doped with 0-100 volume percent of diAnBiz as a guest. The doping ratio is calculated according to the formula: (diAnBiz/ID5+diAnBiz)*100%, and the measurement results are shown in FIGS. 11A-11G and Tables 7-1 and 7-2.

TABLE 7-1

| diAnBiz doping | Voltage (V) @ |
|---|---|
| ratio | 25 mA/cm² |
| 0% | 7.38 |
| 1% | 8.68 |
| 10% | 7.43 |
| 13% | 7.29 |
| 16% | 7.22 |
| 20% | 7.12 |
| 30% | 6.92 |
| 50% | 6.68 |
| 80% | 6.51 |
| 100% | 6.31 |

TABLE 7-2

| diAnBiz doping | max. C.E. | max. P.E. | max. EQE |
|---|---|---|---|
| ratio | (cd/A) | (lm/W) | (%) |
| 0% | 1.27 | 0.67 | 0.64 |
| 1% | 3.45 | 2.58 | 6.99 |
| 10% | 5.61 | 5.05 | 7.44 |
| 13% | 6.57 | 5.91 | 8.29 |
| 16% | 6.65 | 5.97 | 8.12 |
| 20% | 7.16 | 6.52 | 8.04 |
| 30% | 7.45 | 6.46 | 7.72 |
| 50% | 7.56 | 5.70 | 6.83 |
| 80% | 8.11 | 5.55 | 6.75 |
| 100% | 9.15 | 5.75 | 6.73 |

According to the measurement results, it can be found that although the starting voltage (7.29V) is not lowest at the doping ratio of 13%, the current efficiency (6.57 cd/A) is second highest and external quantum efficiency (8.29%) is highest.

Although said descriptions and figures have disclosed preferable embodiments of the invention, it must be understood that possible applications of various additions, many modifications and replacements in preferable embodiments of the invention do not depart from the spirit and scope of the present invention which are as claimed by the appended claims. A person having ordinary skill in the art can realize that the invention can be used in various modifications in structure, arrangements, ratios, material, elements, and components. Therefore, the disclosed embodiments should be regarded as explanations of instead of limitations on the invention. The scope of the invention should be claimed by the appended claims and cover their legal equivalents, and should not be limited to the prior descriptions.

### SYMBOL DESCRIPTION

Substrate 100

First conducting layer 200

Hole transport layer 300

Hole injection layer 310

Hole transfer layer 320

Light emitting layer 400

Electron transport layer 500

Electron transfer layer 510

Electron injection layer 520

Second conducting layer 600

Organic light emitting diode 900

What is claimed is:

1. An anthracene material, having the structure of the following formula (1): 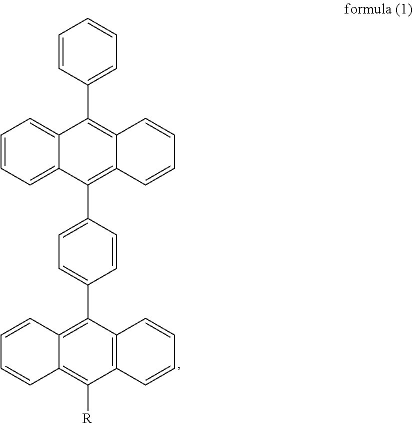
wherein, R is selected from the group consisting of the following groups:

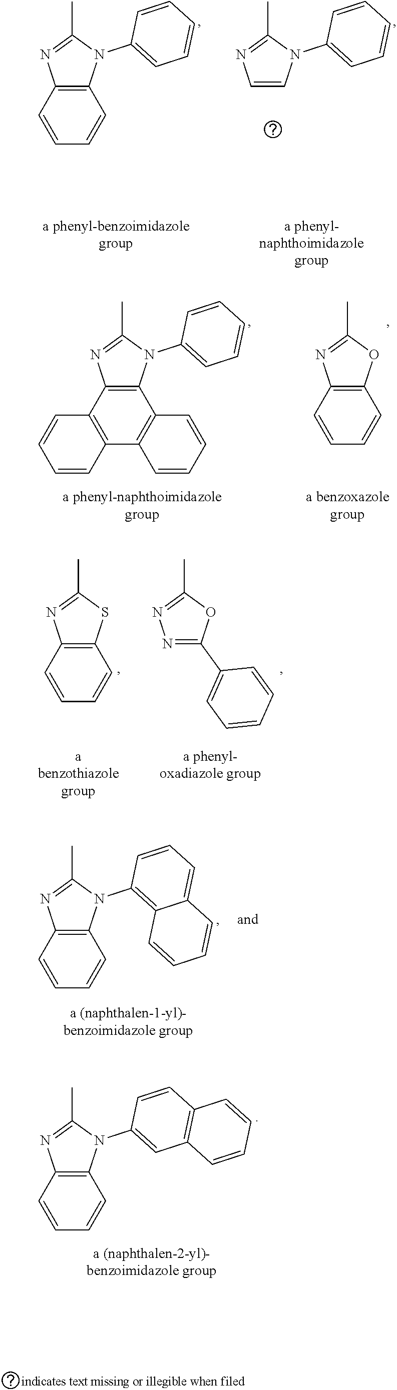

2. An organic light emitting diode, comprising:
a substrate;
a first conducting layer disposed on the substrate;
a hole transport layer disposed on the first conducting layer;
a light emitting layer disposed on the hole transport layer, and containing the anthracene materials with the structure of the following formula (1): 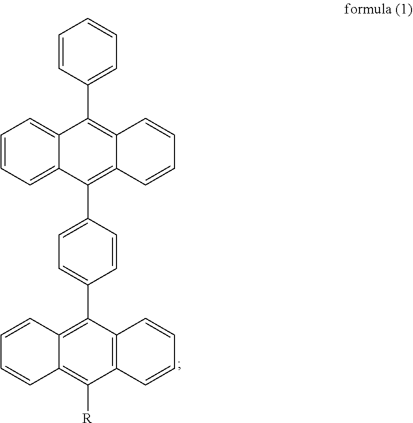
an electron transport layer disposed on the light emitting layer; and
a second conducting layer disposed on the electron transport layer;
wherein, R is selected from the group consisting of the following groups:

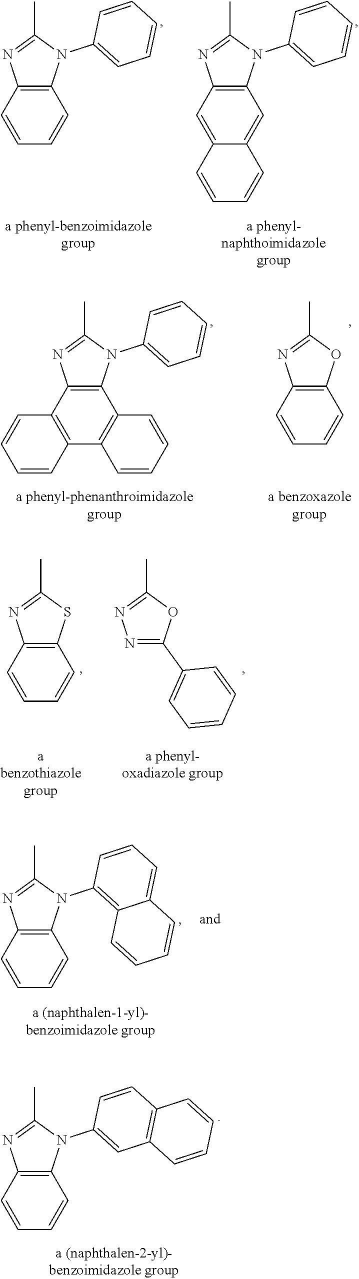

3. The organic light emitting diode according to claim 2, wherein the light emitting layer has a thickness of 200 Å.

4. The organic light emitting diode according to claim 2, wherein the light emitting layer contains:
9,9′-(2-(1-phenyl-1H-benzo[d]imidazol-2-yl)-1,3-phenylene)bis(9H-carbazole) (o-DiCbzBz) as a host; and
1-phenyl-2-(10-(4-(10-phenylanthracen-9-yl)phenyl)anthracen-9-yl)-1H-benzo[d]imid azole (dianthracenebenzimidazole (diAnBiz)) as a guest, wherein the o-DiCbzBz is doped with 13 v/v % of the dianthracenebenzimidazole.

5. The organic light emitting diode according to claim 2, wherein the first conducting layer is an anode.

6. The organic light emitting diode according to claim 2, wherein the hole transport layer includes a hole injection layer and a hole transfer layer disposed on the hole injection layer.

7. The organic light emitting diode according to claim 2, wherein the electron transport layer includes an electron transfer layer and an electron injection layer disposed on the electron transfer layer.

8. A method for manufacturing the anthracene materials by the following equation to produce 9-(4-bromophenyl)-10-phenylanthracene,

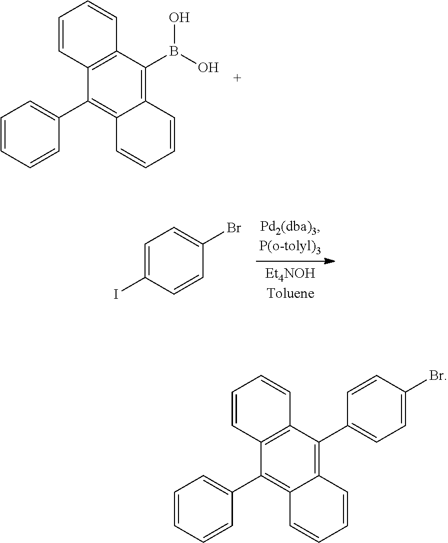 [SMILES: OB(O)c1c2ccccc2c(-c2ccccc2)c2ccccc12 ; Brc1ccc(I)cc1 ; Brc1ccc(-c2c3ccccc3c(-c3ccccc3)c3ccccc23)cc1]

9. A method for manufacturing the anthracene materials by the following equation to produce 10-(4-(10-phenylanthracen-9-yl)phenyl)anthracene-9-carbaldehyde,

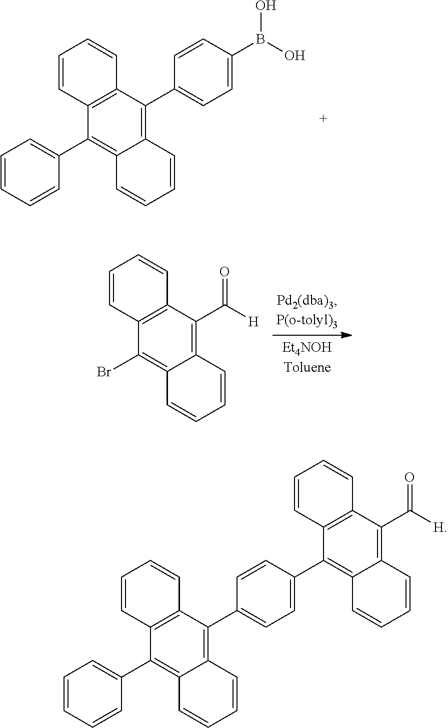

10. A method for manufacturing the anthracene materials by the following equation to produce 1 -phenyl-2-(10-(4-(10-phenylanthracen-9-yl)phenyl) anthracen-9-yl)- 1H-benzo[d] imidazole (dianthracenebenzimidazole), 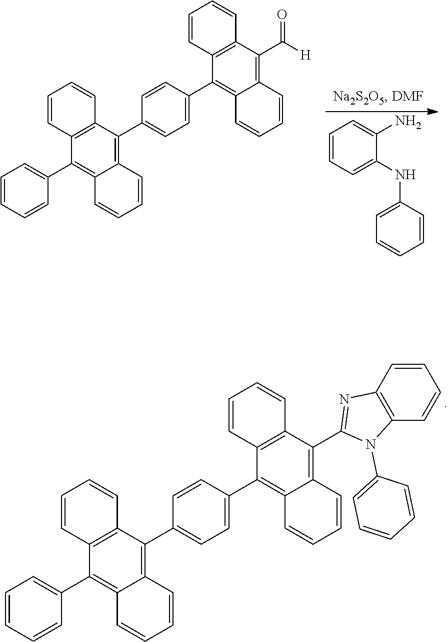 [SMILES: O=Cc1c2ccccc2c(-c2ccc(-c3c4ccccc4c(-c4ccccc4)c4ccccc34)cc2)c2ccccc12 ; Nc1ccccc1Nc1ccccc1 ; c1ccc(-c2c3ccccc3c(-c3ccc(-c4c5ccccc5c(-c5nc6ccccc6n5-c5ccccc5)c5ccccc45)cc3)c3ccccc23)cc1]
